(19) United States
(12) Patent Application Publication
(10) Pub. No.: US 2025/0268820 A1
(43) Pub. Date: Aug. 28, 2025

(54) METHOD FOR MANUFACTURING AN INHALABLE POWDER COMPRISING VORICONAZOLE

(71) Applicant: Zambon S.p.A., Bresso (IT)

(72) Inventors: Laura Zanelotti, Piacenza (IT); Loretta Maggi, Piacenza (IT); Gianluigi Faiella, Verona (IT); Nadia Magi, Gabicce Mare (PU) (IT); Valentina Nicosia, Vicenza (IT); Franco Castegini, Vicenza (IT); Giovanni Caponetti, Piacenza (IT)

(21) Appl. No.: 18/265,463

(22) PCT Filed: Dec. 10, 2021
(86) PCT No.: PCT/EP2021/085197

§ 371 Date: Jun. 6, 2023

(30) Foreign Application Priority Data
Dec. 10, 2020 (IT) 102020000030437

(51) Int. Cl. A61K 9/00; A61K 9/16; A61K 31/506; A61K 47/18

(52) U.S. Cl. CPC A61K 9/0075; A61K 9/1617; A61K 31/506; A61K 47/183

(57) ABSTRACT

The present invention relates to a method for manufacturing an inhalable powder comprising leucine and voriconazole or a pharmaceutically active salt thereof, in substantially crystalline form, in an amount greater than 50% by weight with respect to the total weight of the powder. The method comprises a first step consisting of providing a solution of voriconazole or pharmaceutically active salt thereof and leucine in a suitable vehicle, a second step consisting in drying said powder using the spray drying technique at an outlet temperature from 40 and 75° C. and at a feed rate greater than 10 g/minute and finally collecting the powder obtained.

The present invention relates to formulations of drugs in dry powder form for inhalation administration using a specific inhaler that are highly respirable and stable.

In particular, the present invention relates to a method for manufacturing an inhalable powder suitable for treating pulmonary fungal infections containing drugs belonging to the triazole class, in particular voriconazole.

Inhalation therapy with aerosol preparations is used to administer active ingredients to the respiratory tract, to the mucosal, tracheal and bronchial regions. The term aerosol describes a preparation formed of fine particles or droplets conveyed by a gas (normally air) to the therapeutic action site. When the therapeutic application sites are the alveoli and the bronchioles, the drug must be dispersed as droplets or particles with sizes lower than 5.0 μm in aerodynamic diameter.

When the target is the pharyngeal region, larger particles are more suitable.

Conditions suitable for these treatments are represented by bronchospasm, inflammation, mucosal edema, pulmonary infections and the like.

Currently, administration of drugs into the deep lung is obtained by delivery with inhalation devices such as:
nebulizers, in which the drug is dissolved or dispersed in suspension form and conveyed to the lungs as atomized fine droplets;
pressurized inhalers, through which the drug—once again in the form of droplets of solution or suspension—is conveyed to the deep lung by an inert gas expanded rapidly in air by a pressurized canister;
powder inhalers, capable of dispensing the drug present in the inhaler as micronized dry particles.

In all these cases technological difficulties have been encountered in the manufacture of efficient products, which still today limit the administration of drugs by inhalation.

In the case of inhalation formulations in powder form, these are essentially obtained through the milling/micronization of active ingredients in crystalline form to obtain particles with a diameter generally lower than 5.0 μm, more preferably lower than 2.0 μm. In general, the use of excipients is limited to the resolution of problems related to flow of the powders of the micronized active ingredients dealt with by mixing with lactose with a large particle size used as diluent.

It is evident that the formulation technique based on milling/micronization has several limitations from the point of view of the possibility of processing active ingredients, even with very different chemical and chemical-physical characteristics, ensuring that the final formulation has aerodynamic properties suitable for inhalation administration into the deep regions of the respiratory tract. In this sense, an effective approach for obtaining inhalable powders with good aerodynamic properties is represented by particle engineering, obtainable using the spray drying production technique. According to this technique, the active ingredient and suitable excipients can be combined to form particles whose aerodynamic properties are defined by the composition and by the process conditions used.

Notwithstanding the opportunities offered by particle engineering, this technique is not without formulation difficulties to be overcome. Among the most relevant encountered in the development of inhalable powder products is undoubtedly the need to ensure that the product being developed has sufficient chemical and physical stability over time in relation to atmospheric agents. In fact, these atmospheric agents are capable of determining chemical degradation and/or physical changes in inhalation preparations such as to greatly limit their validity.

The stability of an inhalable product is particularly important in relation to the fact that it must be administered into the deep lung maintaining its physical characteristics for a quantitative penetration of particles or droplets to the deepest regions thereof. Added to this is the fact that the number of excipients currently approved for inhalation administration, and hence acceptable in terms of toxicity in relation to the lung tissue, is extremely limited.

From a clinical point of view, with regard to the main objects of the present invention, pulmonary fungal infections represent an important cause of morbidity and mortality in various types of patients, from patients with asthma through to haemato-oncology patients.

Aspergillus is a genus of fungi of the Trichocomaceae family that comprises around 200 molds. It represents a group of fungi ubiquitous in nature that grow easily in various environments in which there are conditions of high humidity. In suitable conditions, large quantities of spores form, which are then released into the environment, where they remain suspended even for long periods of time.

Among the most common species, Aspergillus fumigatus and Aspergillus flavus are responsible for the infections known as aspergillosis in humans and in animals.

Aspergillus spores are small in size (2.5-3.5 μm in diameter) and can be easily inhaled into the respiratory tract.

If the spores are eliminated immediately, as occurs in the case of healthy individuals, no pathological events occur.

Instead, if colonization takes place, this can have a long or short duration.

The profile of the disease is determined by the characteristics and by the state of health of the individual affected, probably in combination with the size of the inoculum that produces initial colonization.

The invasive disease usually occurs in immunocompromised patients with inhalation as the main infection route. Allergic aspergillosis occurs in patients with asthma, atopy or cystic fibrosis.

Treatment of pulmonary aspergillosis requires the use of systemic drugs. Notwithstanding this, the distribution of therapeutic agents from the bloodstream to the tissue sub-compartments such as the lungs is often characterized by considerable variability and the concentrations of drug in the target site are often very different with respect to those measured in the plasma.

Moreover, some low and sub-optimal concentrations in the target site could be responsible for some cases of ineffectiveness of antifungal active ingredients.

Triazole antifungal agents have a characteristic structure as they contain three nitrogen atoms in the base ring. The active ingredients in current clinical use include itraconazole, fluconazole, voriconazole and posaconazole.

These compounds are all distinct in terms of chemical structure and molecular weight, lipophilicity and metabolism; these differences have an important impact on their pharmacokinetics and pharmacodynamics.

In fact, said chemical-physical properties determine the speed and the degree of penetration and distribution in the various tissues of the body as well as the relative bioavailability in tissues, organs and biological fluids.

Fluconazole, which is an antifungal triazole, is not active against invasive aspergillosis.

Itraconazole is approved for systemic use for the treatment of invasive aspergillosis in patients who are unresponsive or intolerant to standard antifungal therapy.

Posaconazole is approved by the FDA for the prevention of invasive aspergillosis.

Voriconazole is approved by the FDA for the primary treatment of invasive aspergillosis and is currently considered the standard of therapy for this disease; voriconazole is formulated in oral tablets or in intravenous solution in the form of sulfobutylether cyclodextrin inclusion complex. Pulmonary infections start in the airways. For this reason, in the case of antifungal agents used for the prophylaxis or treatment of infections of the airways obtaining high concentrations at the level of the epithelial lining fluid and of the alveolar macrophages is crucial. Post-mortem studies conducted on homogenates of lung tissue of patients treated with voriconazole have shown concentrations of voriconazole comparable to those measured in the plasma.

Healthy volunteers treated with intravenous loading doses of voriconazole followed by oral doses of 200 mg twice a day, showed an ELF/plasma concentration ratio of 11. (Felton T., Troke P F., Hope W W. 2014. Tissue penetration of antifungal agents. Clin Microbiol Rev. 27(1): 68-88.)

The bioavailability of voriconazole following oral administration to patients who have not undergone transplant is 96%.

Instead, in the case of intravenous administration, obtained through an initial loading dose followed by 3 doses of 4 mg/kg every 12 hours, the literature has reported a variable ELF/plasma concentration ratio ranging from 6 to 9 and a variable alveolar macrophage/plasma concentration ratio ranging from 3.8 and 6.5.

In the case of itraconazole, this exhibited an ELF exposure of approximately ⅓ of the plasma concentration in healthy volunteers, while the concentration in the alveolar cells was more than double with respect to the plasma concentration.

In other cases, itraconazole concentrations in the fluid obtained from bronchoalveolar lavage and from the lung tissue in the airways were 10 times lower than those measured in the plasma. In post-mortem samples obtained from 4 hematological patients, the mean lung tissue/plasma concentration ratio of itraconazole was reported as ranging from 0.9 to 7.

Therefore, the results reported convincingly show that it is possible to obtain, both after oral administration and administration by injection, even relatively high concentrations of triazole active ingredients with antifungal action at the level of different elements of the respiratory tract, including the epithelial fluid, the alveolar macrophages and the tissue itself. However, this positive effect of high concentration is not achieved without involving other important body systems.

Firstly, the extended residence time of the active ingredients with greater lipophilicity and the risk of accumulation in the various organs at concentrations much higher than those in the plasma must duly considered.

In the case of voriconazole, following oral or intravenous administration its hepatic metabolism represents an element of concern, as only 5% of the drug is excreted unchanged in the urine. Voriconazole is associated with a non-linear pharmacokinetic profile, a maximum concentration in the plasma and an area under the plasma curve (AUC) that increases in a manner that is not proportional to the increase in the dose administered.

Voriconazole is a metabolic substrate and inhibitor of cytochromes CYP2C19, CYP2C9 and CYP3A4. In the case of patients being treated with different drugs for another disease, very careful evaluation of potential interactions with these drugs must be carried out.

The treatment of the invasive aspergillosis with voriconazole involves, in the first 24 hours, an initial loading dose of 6 mg/kg iv every 12 hours, followed by doses of 4 mg/kg every 12 hours.

These doses are higher than those routinely used orally (200 mg every 12 hours).

In the case of pediatric patients, due to their accelerated metabolism and rapid clearance, the doses of voriconazole could even be higher.

The profile of the possible side effects of voriconazole include temporary visual disturbances (photopsia), hepatotoxicity, which is manifested through an increase in serum bilirubin, in alkaline phosphatase and in hepatic aminotransferase and can influence the dose to be administered; cutaneous eruptions, visual hallucinations and other side effects.

For all the aforesaid reasons, it is evident that a treatment with voriconazole that uses the inhalation route would be capable of optimizing administration to the target organ with a drastic reduction in the dose administered, as it is no longer necessary to distribute the active ingredient throughout the body.

Specifically, the chemical-physical properties of voriconazole and the degree of lipophilicity with respect to itraconazole suggest that once the active ingredient is administered directly into the lung it would be capable of being distributed at a high concentration both in the epithelial lining fluid and at the level of the lung tissue and possibly also of the macrophages. The fact that this active ingredient, with respect to itraconazole, is not inclined to accumulate in the various tissues treated must also be considered important.

Allergic Bronchopulmonary Aspergillosis (ABPA) is not an invasive disease, but rather a disease characterized by hypersensitivity towards Aspergillus. Therapeutic indications differ greatly with respect to those for invasive aspergillosis. The aim of the therapy for ABPA is directed at the prevention and at the treatment of acute exacerbations and at prevention of the fibrotic end stage that can develop in the patient. Systemic corticosteroids are the drugs of choice for this therapy. The dose initially prescribed is 0.5 mg/kg/day of prednisone (or other equivalent corticosteroid), with a progressive decrease of the dose starting from the time in which the symptoms start to improve.

Less severe exacerbations can be managed through the use of corticosteroids and bronchodilators through inhalation.

In the case of acute exacerbations, a recommended therapeutic cycle consists of a dose of prednisone of 0.5-1.0 mg/kg/day for 1-2 weeks, followed by a dose of 0.5 mg/kg on alternate days for 6-12 weeks following clinical remission and further reduction of the dose to the doses originally used in the period prior to exacerbation.

Exacerbations in asthma, in the light of this management strategy, require chronic therapies with doses of corticosteroids normally higher than 7.5 mg/kg/day.

It must be noted that ABPA is particularly critical in patients with cystic fibrosis with the disease prevailing in 10% of all cystic fibrosis patients.

In view of the fact that severe lung damage can also occur in asymptomatic patients, it is important to carefully monitor the level of serum IgE at regular intervals (every 1-2 months). Periodic monitoring of respiratory function and chest X-rays are also recommended. If lung the presence of infiltrates, mucoid elements, fibrosis, worsening of bronchiecstasis or physiological deterioration is found, adaptation of the therapy with corticosteroids is recommended.

In these patients, in association with the steroid, the introduction of a twice daily 200 mg oral dose of itraconazole for up to 6 months has been proposed, obtaining good results which allow a significant reduction in the use of oral corticosteroids.

Inhalation administration of antifungal drugs represents a very attractive option, as using this route is theoretically possible to reach very high local concentrations of the drug with minimal systemic exposure, particularly important especially in the case of some of these agents for which systemic administration is associated with significant side effects.

Colocalization of the drug and of the pathogenic agent in a tissue or an organ is in fact the ideal way to make a therapeutic treatment effective against an infectious agent.

Unlike the oral and parenteral methods of administering drugs, which require the diffusion thereof to reach the site of the infection, the administration of drugs through inhalation conveys anti-infective agents directly into the respiratory system.

Consequently, administration through inhalation can maximize their effectiveness and limit systemic toxicity.

In the case of inhaled anti-infective drugs, to allow them to be effective, administration must be optimized in order to obtain therapeutic concentrations at the site of the infection in the deepest regions of the respiratory tract.

Differences in the administration technique can cause considerable variations, even greater than 100%, of the dose effectively administered.

Two key aspects related to the direct administration of antimicrobial agents into the respiratory tract are linked to the characteristics of the aerosolized particles and to the aerosol administration methods. The physical properties of antimicrobic formulations can have significant effects on administration of the drug, as well as having an impact on the tolerability by the patient.

For this reason, very few anti-infective therapies have been specifically formulated for inhalation administration and, in some cases, injectable preparations are administered through nebulizers in the form of aerosol.

At times these formulations are not optimized for aerosol administration and can have physical properties (i.e. particle size distribution, viscosity, surface tension, osmolality, tonicity, pH) that make their administration difficult and/or harmful, in some cases causing side effects such as coughing and bronchoconstriction.

In general, a drug in liquid formulation to be administered via aerosol should have an osmolality from 150 to 1200 mOsm/kg, a sodium content in the range from 77 to 154 mEq/L and a pH from 2.6 to 10.

These characteristics of the formulation are not always present even in intravenous preparations.

Moreover, preservatives, such as phenols and sulfites, which are found in some parenteral preparations can contribute to producing coughing and irritation of the airways, as well as bronchoconstriction.

The primary property for deposition in the airways and in the alveoli is the aerodynamic diameter of the particles (or droplets) of the aerosol.

The parameter of reference that characterizes the aerodynamic size distribution of the particles of an aerosol for inhalation is the MMAD, or Mass Median Aerodynamic Diameter.

In view of positive clinical elements found with triazole antifungal active ingredients, administered orally and intravenously for the treatment of different types of aspergillosis, the potential use of inhaled Voriconazole in the treatment of various forms of aspergillosis, including invasive aspergillosis and ABPA must be considered.

Preliminary studies with promising effects have been published on the intravenous formulation of Voriconazole administered through inhalation using a nebulizer, in 3 different cases of invasive aspergillosis in which systemic therapy with voriconazole had been suspended due to adverse side effects that had become unacceptable.
(Hilberg O., Andersen C U., Henning O., Lundby T., Mortensen J., Bendstrup E.; Remarkably efficient inhaled antifungal monotherapy for invasive pulmonary aspergillosis. Eur. Resp. J. 40 (1) 271-273)

As already mentioned above, the manufacture of an inhalation formulation obtained by converting the product available for intravenous administration is not a technically acceptable route, for the reasons already stated.

In particular, the inclusion of voriconazole in a cyclodextrin to make this ingredient soluble in water is not approved from a regulatory point of view.

For this reason, a desirable inhalation formulation containing a triazole antifungal agent capable of effectively and safely treating forms of lung infection caused by Aspergillus fumigatus, and fungi of the same genus, can be produced through the preparation of an inhalable powder comprising voriconazole and provided with suitable aerodynamic characteristics and sufficient physical and chemical stability.

As confirmation of the technical difficulties for the formulation that the person skilled in the art has to face, it should be mentioned that triazole antifungal drugs and, in particular, voriconazole, are active ingredients that have been known since the last century, for which use by inhalation was proposed starting in the 1990s.

However, to date there is still no drug suitable for pulmonary administration comprising said active ingredients available on the market, which has, therefore, been approved by the competent regulatory authorities.

The scientific and patent literature describe inhalable powders comprising antifungal drugs potentially useful for the treatment of pulmonary fungal infections.

US 2019/0167579 describes a dry powder comprising itraconazole in amorphous form, in an amount from 45 to 75%, which can be used to treat pulmonary Aspergillosis. However, the powder described could have problems of physical and chemical stability, in particular in conditions of high temperatures and humidity, due to the prevalently amorphous solid state of the powder, which could influence the performance and stability of this powder over time.

WO 2018/071757 describes a dry pharmaceutical composition for inhalation comprising a crystalline antifungal drug in the form of sub-particles. The particles of the final powder formulation are produced through the initial preparation of a stabilized suspension of nanoparticles of the antifungal active ingredient, followed by a spray drying process. This formulation has a production process that is difficult to transfer from pilot scale to industrial scale. It must be noted that the experimental part of the international patent application is aimed at the development of dry powders comprising the active ingredient itraconazole.

EP2788029B1 describes pharmaceutical compositions for inhalation containing triazoles in amorphous form. These compositions have a low active ingredient load, which together with the physical form described exposes the formulation to problems of stability and at the same time limit its use in some pulmonary diseases. Moreover, some specific excipients can be present in the formulation, such as polyols and sugars, which could alter the stability of the active ingredient. It must be noted that the experimental part of the patent is directly exclusively at the development of dry powders comprising the active ingredient itraconazole.

In the light of the considerations set forth above, it would be advantageous to manufacture a pharmaceutical composition for inhalation in dry powder form comprising triazoles, and in particular voriconazole, which is stable and can be easily administered with common dry powder inhalers, while at the same time remaining easy to produce.

It would also be desirable to obtain a method for the preparation of pharmaceutical compositions for inhalation comprising voriconazole that is applicable on an industrial scale, providing a stable and deliverable product that minimizes manufacturing costs.

At the current state of the art, the problem of providing an inhalation formulation of drugs comprising voriconazole that is stable and can be administered with common dry powder inhalers, maintaining characteristics of high deliverability and respirability, and which can be manufactured industrially with a process that is advantageous from an economic point of view, has still not been solved, or has been solved in an unsatisfactory manner.

The first aspect of the present invention is therefore to provide a method for the preparation of an inhalable powder comprising voriconazole or a pharmaceutically active salt thereof, in substantially crystalline form, and in amount greater than 50% by weight with respect to the total amount of the powder.

In particular, the present invention relates to a method for manufacturing an inhalable powder comprising leucine and voriconazole or a pharmaceutically active salt thereof, in substantially crystalline form and in an amount greater than 50% by weight with respect to the total amount of the powder, said method comprises the steps of:
a) providing a homogeneous solution of voriconazole or its pharmaceutically active salt and leucine in a suitable vehicle;
b) spray drying said powder at an outlet temperature from 40 to 75° C. and at a feed rate greater than 10 g/minute;
c) collecting said powder.

A further aspect of the invention is represented by an inhalable powder obtained by the preparation method described above.

According to the present invention, the term “inhalable” means that the powder is suitable for pulmonary administration. An inhalable powder can be dispersed and inhaled by means of a suitable inhaler, so that the particles of which it is composed can penetrate into the lungs to reach the alveoli in order to perform the pharmacological characteristics of the active ingredient of which it is composed. Particles with an aerodynamic diameter lower than 5.0 μm are normally considered inhalable.

In an aspect of the invention the active ingredient is present in crystalline form; i.e., voriconazole has a specific solid state and an orderly rearrangement of the structural units which are arranged in fixed geometrical models.

The term “substantially crystalline” according to the present invention means that the percentage of the active ingredient, voriconazole, in the crystalline solid state ranges from 51-100%, preferably from 70-100% and even more preferably from 90-100% with respect to its total amount in the powder.

Preferably, the powder obtained by the method according to the present invention has a fine particle fraction (FPF) greater than 40%, preferably greater than 50%.

The term “fine particle fraction (FPF)” means the fraction of powder, with respect to the total amount of powder delivered by an inhaler, which has an aerodynamic diameter (aed) lower than 5.0 μm. The term “delivered fraction (DF)” means the fraction of active ingredient delivered, with respect to the total loaded. The characterization test that is conducted to evaluate the properties of the powder is the Next Generation Impactor (NGI) test as described in the current edition of the European Pharmacopoeia. According to the present invention, the conditions for performing this test consist in subjecting the powder to aspiration through the inhaler such as to generate a flow of 60±2 liters/min. This flow in the case of the inhaler model RS01 (Plastiape, Osnago IT) is obtained by generating a pressure drop of 2 Kpa in the system.

According to the present invention, pharmaceutically active salts of voriconazole are, for example, acetate, sulfate, citrate, formate, mesylate, nitrate, sulfate, hydrochloride, lactate, valinate and the like.

In order to obtain a stable and pharmaceutically active powder for inhalation, voriconazole, or a pharmaceutically active salt thereof, is preferably present in an amount from 50 to 85% by weight with respect to the total amount of the powder.

Even more preferably, voriconazole, or its pharmaceutically active salt, is present in an amount equal to 70% by weight with respect to the total amount of the powder.

In the preferred particle size for this powder, at least 90% of the size distribution (X₉₀) is lower than 10 μm, preferably lower than 7 μm, in order to increase the surface area optimizing lung deposition.

In an even more preferred embodiment, at least 90% of the size distribution (X₉₀) is from 4.5 to 7 μm.

According to the present invention, the powder obtained with the method described has a Mass Median Aerodynamic Diameter (MMAD) of the particles delivered equal or lower than 5 μm, preferably from 3 to 4.5 μm.

In an even more preferred embodiment, the powder according to the present invention has a Mass Median Aerodynamic Diameter (MMAD) of the particles delivered from 3.5 to 4.5 μm.

Preferably, said leucine is present in an amount greater than 10% by weight with respect to the total amount of the powder, even more preferably in an amount from 14 to 49% by weight with respect to the total amount of the powder; and even more preferably in an amount from 25 to 35% by weight with respect to the total amount of the powder.

Leucine is preferably in non amorphous form, more preferably in crystalline form.

The powder obtained according to the method described herein is a substantially dry powder, i.e., a powder that has a humidity content below 10%, preferably below 5%, more preferably below 3%. This dry powder preferably does not have water in amounts sufficient to hydrolyze the active ingredient making it inactive. The amount of humidity present in the composition is controlled by the presence of leucine which, thanks to its hydrophobic characteristics, limits its content both in the production phase of the powder and in the subsequent handling phases.

Preferably, in said step a) of the method according to the present invention a surfactant is present, preferably in a solution.

Preferably, said surfactant is present in an amount from 0.2 to 2.0% by weight with respect to the amount of each powder, preferably in an amount from 0.4 to 1.2% by weight with respect to the amount of each powder, even more preferably 1%.

The surfactant of the pharmaceutical composition according to the invention can be selected from the various classes of surfactants for pharmaceutical use.

Surfactants that can be used in the present invention are all those substances characterized by medium or low molecular weight that contain a hydrophobic portion, which is generally readily soluble in an organic solvent but poorly soluble or insoluble in water, and a hydrophilic (or polar) portion, which is poorly soluble or insoluble in an organic solvent but readily soluble in water. Surfactants are classified according to their polar portion; therefore, surfactants with a negatively charged polar portion are defined as anionic surfactants while cationic surfactants contain a positively charged polar portion. Surfactants with no charge are generally defined nonionic while surfactants that contain both a positively charged group and a negatively charged group are called zwitterionic. The salts of fatty acids (better known as soaps), sulfates, sulfate ethers and sulfate esters represent examples of anionic surfactants. Cationic surfactants are frequently based on polar groups containing amino groups. The most common nonionic surfactants are based on polar groups containing oligo-(ethylene-oxide) groups. Zwitterionic surfactants are generally characterized by a polar group consisting of a quaternary amine and a sulfuric or carboxylic group.

Specific examples of this application are represented by the following surfactants: benzalkonium chloride, cetrimide, docusate sodium, glyceryl monooleate, sorbitan esters, sodium lauryl sulfate, polysorbates, phospholipids, bile salts.

Nonionic surfactants such as polysorbates and polyoxyethylene and polyoxypropylene block copolymers, known as “Poloxamers” are preferred. Polysorbates are described in the CTFA International Cosmetic Ingredient Dictionary as mixtures of sorbitol and sorbitol anhydride fatty acid esters condensed with ethylene oxide. Particularly preferred are nonionic surfactants of the series known as “Tween”, in particular the surfactant known as “Tween 80”, a polyoxyethylene sorbitan monooleate available on the market.

The presence of a surfactant is useful to ensure the reduction of electrostatic charges found in formulations without it, flow of the powder and maintenance of the homogeneous solid state without initial crystallization.

According to the present invention in said solution pursuant to step a) of the manufacturing method, one or more excipients can advantageously also be present in the vehicle used in this solution, in particular excipients suitable for inhalation administration.

These excipients are preferably sugars, such as lactose, mannitol, sucrose, trehalose, maltodextrin and cyclodextrin; fatty acids; esters of fatty acids; lipids, preferably phospholipids, such as natural and synthetic sphingophospholipids and natural and synthetic glycerophospholipids including diacyl phospholipids, alkyacyl phospholipids and alkenylacyl phospholipids; amino acids; and peptides such as di-leucine and tri-leucine or hydrophobic proteins.

According to the present invention, the vehicle in which voriconazole or a pharmaceutically active salt thereof, and leucine are dissolved in the first step a) is any solvent in which the active ingredient and the excipient are soluble, such as organic solvents, aqueous solvents and/or mixtures thereof.

Preferably, the vehicle according to the invention consists of a hydroalcoholic mixture.

Even more preferably, the vehicle is a mixture of water and alcohols, where said alcohols are advantageously selected from the group consisting of methanol, ethanol, 1-propanol, 2-propanol, 2-methyl-1-propanol, 1-butanol, 2-butanol, 3-methyl-1-butanol, 1-pentanol and the like, alone or in a mixture.

Preferably, the alcohols are in a ratio with water from 70/30 and 30/70 v/v, and even more preferably in a ratio of 60/40 v/v.

Preferably, the alcohol is ethyl alcohol and therefore the preferred vehicle is a hydroalcoholic mixture of water and ethyl alcohol.

As is well known, spray drying is a technique that allows powders with uniform and substantially amorphous particles to be obtained from solutions of active ingredients and excipients in an appropriate solvent or mixture of solvents.

This technique consists of a series of operations, illustrated below:
preparing a first phase in which an active ingredient and any excipients are dissolved or dispersed in a suitable liquid medium;
drying said phase in controlled conditions to obtain a dry powder with particles with a size distribution having a mean diameter lower than 10.0 μm;
collecting said dry powder.

The first phase can be either a suspension of the active ingredient in a liquid medium, aqueous or not, or a solution of the active ingredient in a suitable solvent.

Preparation of a solution is preferred, and the organic solvent is selected from those miscible with water.

The drying operation consists in eliminating the liquid medium, solvent or dispersant, to obtain a dry powder having the desired size characteristics. The characteristics of the nozzle and the process parameters are selected so that the liquid medium is evaporated from the solution or suspension and a powder with the desired particle size is formed.

In a preferred aspect of the invention, step a) of the manufacturing method is advantageously composed of three distinct sub-steps:
a1) providing an aqueous solution of leucine, optionally surfactant and optionally soluble excipient;
a2) providing a solution of voriconazole in a suitable organic solvent;
a3) mixing the solution indicated in sub-step a1 with the solution indicated in sub-step a2.

In this way, a stable and homogeneous solution of voriconazole, leucine and any other optional components is prepared, avoiding the formation of precipitates so that it can be easily dried through the spray drying technique.

In order to obtain a powder with the desired characteristics according to the invention, the feed rate of the spray dryer must be greater than 10 g/minute, preferably greater than 15 g/minute, even more preferably equal to or greater than 20 g/minute. In this way a powder is obtained comprising voriconazole and leucine in substantially crystalline form, contrary to what normally occurs with the spray drying technique as described above.

The maximum feed rate at which it is possible to operate in order to obtain a powder with the desired characteristics according to the invention, is dictated by the type of spray dryer used, i.e., an industrial scale or a pilot scale spray dryer. Therefore, the maximum feed rate is currently 200 g/minute, but there are no limits if larger machinery were to be used.

In a preferred embodiment, the feed rate of the spray is in the range from 90 to 200 g/minute. In an even more preferred embodiment, the feed rate is in the range from 140 to 180 g/minute For the same reasons set forth above, the outlet temperature should be from 40 to 75° C., preferably from 50 to 70° C.

In an even more preferred embodiment, the outlet temperature is in the range from 55 to 70° C. The term outlet temperature according to the present invention means the temperature of the product already dried after exiting from the drying chamber and before entering the cyclone separator.

The term inlet temperature according to the present invention means the temperature the solution encounters when it exits from the nozzle of the spray dryer.

According to the present invention, the inlet temperature is from 80 to 140° C. preferably from 90 to 120° C.

As already described in depth above, in order to obtain an inhalation formulation in powder form containing voriconazole with a preparation method conducted on a larger scale than the scale used in the laboratory, for example pilot and industrial scale, it is essential for the powder to be given different specific characteristics by combining not only essential aspects of pharmaceutical performance, such as aerodynamic performance for the delivery of the largest possible amount of drug to the deep lung regions, but also aspects of quality of the product and of efficient industrial manufacture. For this reason, the ideal preparation should be characterized simultaneously by:
the possibility of administering high dosages in a single dose;
a reduced aerodynamic size of the particles;
the chemical and physical stability of the formulation;
a high efficiency of the production process in terms of yield.

With reference to the administration of high dosages by inhalation, as in the case of voriconazole, the active ingredient selected, this must be considered relevant due to the fact that it is conventionally administered orally or parenterally at dosages of no less than 200 mg/dose. In the case of inhalation administration in powder form, the dose is significantly lower, around 10-40 mg/dose, which in any case represents a relatively high dosage in relation to the inhalation administration route.

With regard to the possibility of administering high dosages by inhalation in powder form, this can potentially be achieved by managing to introduce therein percentage portions of active ingredient of at least 50% by weight, in order to prevent the inhalation of large amounts of powder from stimulating a cough reflex in the patient. The spray drying manufacturing technique generally makes it possible to produce engineered particles of powder combining suitable amounts of active ingredients and excipients that perform the function of facilitating particle separation or promoting the formation of low density structures. These facilitating effects are clearly better in relation to the percentage of excipient that can be added to the composition of the powder. In the case of an active ingredient such as voriconazole, characterized by low solubility in aqueous solvent, initially it has a high propensity not to form homogeneous particles with different excipients by spray drying and not to associate with these in a homogeneous structure, even more so if there is a high voriconazole content in the composition, as desired. Therefore, the powder obtained could be characterized by a distribution of particles each of which is not perfectly homogeneous in composition with respect to the solution of the initial components. The final result expected is however of a homogeneous powder in terms of content of active ingredient with respect to the initial solution and to the excipients introduced. The cause of this possible lack of homogeneity of the single particles of powder is to be found in the propensity of the active ingredient voriconazole to form particles or crystalline structures during the spray drying process. However, in order to ensure a final homogeneity of the powder it is necessary to use process conditions that favor this homogeneity. More specifically, it has been found that conditions with drying temperatures that are too high can cause, in the case of mixtures of different components, diversified drying of these components during the process.

With regard to the aerodynamic size of the particles of powder, such as to ensure a respirability thereof of over 50% of the dose administered to the patient, the spray drying production technique allows the engineering of aerodynamically fine particles (mass median aerodynamic diameter (MMAD) lower than 5.0 μm) consisting of high amounts of Voriconazole, associated with excipients capable of ensuring the formation of particles of powder easily dispersible when it is subjected to an air flow such as the one generated by a powder inhaler during inhalation. This formulation approach, in the case of a formulation containing Voriconazole, unlike other cases reported in the literature for different inhalation powders, does not require the use of particularly high percentages of excipients in the formulation and allows amounts of Voriconazole of over 50% to be contained in the composition.

With reference to the chemical and physical stability of the powder, it must remain stable for 24 months at temperature conditions of 25° C.

Consequently, the manufacture of an inhalable powder that is chemically and physically stable must reconcile the need for stability of the active ingredient used with the need to ensure adequate aerosol performance in terms of delivery to the deep lung.

An ideal approach for obtaining chemical and physical stability is represented by the manufacture of a dry powder of voriconazole containing high amounts of this active ingredient in combination with a pharmaceutical excipient, which can be administered by inhalation and which has a high level of local tolerability in relation to the lung epithelium. In a similar way to voriconazole, for spray drying the excipient must be able to arrange itself into a preferentially crystalline solid state during the process. The formation of an inhalable powder in which, after spray drying, the majority of the components can be obtained in crystalline form is able to guarantee the prolonged physical and chemical stability thereof also in conditions of high temperature and humidity. The powder obtained can comprise particles formed of voriconazole and excipients in which each single particle has a composition equivalent to the composition subjected to the spray drying process. It is also acceptable for the final powder to reflect, in its total composition, the proportions of voriconazole and excipients subjected to the spray drying process but for it to be formed of particles that individually have a different composition from one another.

With reference to the production yield of the process, this cannot be underestimated as it is theoretically possible to produce particles containing voriconazole that can be administered by inhalation with high respirability but obtained through a production process that is not particularly efficient. This is without doubt the case of spray drying equipment for use in the laboratory. A yield of the spray drying process of the powder of at least 50 g of powder produced in 6 hours should be the target of reference of a pilot or industrial production process. These production rates can only be achieved through the spray drying of large amounts of solution in the unit of time. Purely by way of indication, an efficient production process should be able to treat at least 20 grams of solution per minute.

In order to better illustrate the present invention some examples are set down below.

### EXAMPLES

Some examples of a method of manufacturing an inhalable powder comprising voriconazole in substantially crystalline form according to the present invention are described below.

### Preparation of the Powders.

As described above, the powders containing the active ingredients were obtained by spray drying,

For the formulations described the solvents used were water and ethyl alcohol in a fixed ratio of 54/45 (p/p). The concentration of dissolved solids was 1% p/v.

For preparation of the powder two solutions were prepared: an aqueous solution containing the excipients Leucine and surfactant in a solution, and an alcohol solution containing the active ingredient Voriconazole. The aqueous portion was then added to the alcohol solution slowly at room temperature to obtain a single clear hydroalcoholic solution, taking care to avoid precipitation of any of the components.

The hydroalcoholic solution thus obtained was processed by mean of:
a GEA NIRO PSD1 Spray Dryer, using a closed cycle, setting the following process parameters:
bi-fluid nozzle with a diameter of 0.5 mm for delivery of the solution, with gas outlet nozzle cup having a diameter of 5 mm
atomizing gas: nitrogen
atomizing pressure: 3 bar
drying gas: nitrogen
drying gas flow rate: 80 kg/h
inlet temperature: 90-120° C.
feed speed: 20 g/min
Powder collection system: cyclone separator
Outlet filter system: Teflon membrane filter.
GEA NIRO PSD2 Spray Dryer using a closed cycle, setting the following process parameters:
bi-fluid nozzle with a diameter of 0.5 mm for delivery of the solution, with gas outlet nozzle cup having a diameter of 5 mm
atomizing gas: nitrogen
atomizing pressure: 4 bar
drying gas: nitrogen
drying gas flow rate: 360 kg/h
inlet temperature: 98-103° C.
feed speed: 100-120 g/min
Powder collection system: cyclone separator
Outlet filter system: Teflon membrane filter.

At the end of the drying process, the powders were packaged immediately after production in polyethylene bags, in turn stored in heat-sealed aluminum bags.

### Characterization of the Powder: Particle Size Analysis.

The powders obtained were characterized in terms of dry particle size using a Sympatec HELOS/BR Laser Diffraction device, capable of analyzing the particle size, equipped with a RODOS/L dispersion unit for powder analysis, associated with the ASPIROS/L system for automatic loading of the sample.

The instrument was calibrated with reference material and prepared following the instructions provided in the instrument user manual.

### Analysis Procedure:

The product was sampled in a specific sample holder (vial) for Aspiros and analyzed.

The dispersion gas used was compressed air suitably cleansed of particles.

The method used for Particle Size Distribution analysis was the following:
analysis instrument: Sympatec HELOS/BR Laser Light Diffraction device
lens: R1 (0.1-35 μm)
sample dispersion system: RODOS/L
sample feed system: ASPIROS/L
dispersion pressure: 3 bar, with auto-adjustment of the vacuum pressure
signal integration time: 10.0 s
duration of the reference measurement: 10 s
measurement valid in the range of concentrations of channel 20 from 1.5% to 50%
software version: PAQXSOS 3.1.1
calculation method: FREE

All analyses were conducted at room temperature and room humidity.

Size analysis returns the diameter values respectively of 10% of the population (X₁₀); 50% of the population (X₅₀); 90% of the population (X₉₀) and the volume median diameter (VMD) of the population of particles in the sample of powder.

### Characterization of the Powder: Determination of Titer and Related Substances.

The HPLC (High Performance Liquid Chromatography) analysis method was used to determine the content of active ingredient (titer) and of the related substances.

The analysis method used is characterized by the following parameters:
solvent: 70/30 methanol/water
mobile phase: methanol/phosphate buffer pH 7.5 10 mM
gradient elution

| Time | | % buffer |
|---|---|---|
| (min) | % Methanol | pH 7.5 |
| 0 | 70 | 30 |
| 1.5 | 70 | 30 |
| 2.5 | 90 | 10 |
| 5.5 | 90 | 10 |

+2 min post time

flow rate: 1 ml/min
injection volume: 2 μl
analysis column: Agilent Poroshell 120 EC-C18, 100 mm×4.6 mm, 2.7 μm
column temperature: 45° C.
wavelength: 254 nm
retention time: 1.8 min

A model 1200 HPLC Agilent with model G1315C diode array type detector was used for the analyses.

The samples for analysis of the content in active ingredient were obtained by dissolving in the solvent an amount of powder such as to obtain a concentration from 50 μg/ml to 90 μg/ml of Voriconazole, as per the reference solution.

The samples for analysis of the impurities were obtained by dissolving in the solvent an amount of powder such as to obtain a concentration from 500 μg/ml to 900 μg/ml of Voriconazole.

The reference solution was injected three consecutive times before the sample, to determine the precision of the system, expressed as relative standard deviation percentage (RSD %), which must be lower than 2%.

The active ingredient content is obtained by calculating the ratio of the area with respect to the reference solution at known concentration. The degradation of the product is calculated as the ratio between the sum of the areas of the analysis peaks corresponding to the degradation products, corrected for each response factor and the area of the active present in the sample. All the analysis peaks with an area greater than 0.1% with respect to the area of the active were included in the sum of the degradation products.

Characterization of the Powder: Respirability Test with NGI (Next Generation Impactor).

The Next Generation Impactor (NGI) is a powder impactor, described in pharmacopoeia (EP; USP), used to measure the aerodynamic diameter of particles of powder dispersed in the air in the form of aerosol. An inhalation formulation, dispensed by a suitable inhaler and conveyed into the instrument by aspiration, is deposited in the various stages of the impactor, positioned in series, according to its aerodynamic characteristics, which depend on particle size, density and form. Each stage of the NGI corresponds to a range of aerodynamic particle sizes of the powder deposited therein, determined by HPLC quantitative analysis of the active ingredient present. Through quantitative active ingredient determination in each stage, the aerodynamic size distribution of the powder is obtained and the median aerodynamic diameter and respirable fraction, defined by the European Pharmacopoeia as the fraction having an aerodynamic diameter <5.0 μm, can be calculated.

For the respirability test, the powders of the formulations of the examples were divided into size 3 HPMC capsules and dispensed through a model 7 single dose RS01 powder inhaler, code 239700001AB (Aerolizer-Plastiape S.p.A.).

The instrument was assembled according to the instructions for use and following the indications of the European Pharmacopoeia.

In order to conduct the test, the delivery of a single powder capsule is sufficient for each respirability test. The tests were conducted at a flow rate of 60 lpm for 4 seconds deriving from a pressure drop of 2 KPa in the system.

The following aerodynamic diameter cut-offs correspond to this flow rate for each stage of the NGI.
stage 1: >8.06 μm
stage 2: from 8.06 μm to 4.46 μm
stage 3: from 4.46 μm to 2.82 μm
stage 4: from 2.82 μm to 1.66 μm
stage 5: from 1.66 μm to 0.94 μm
stage 6: from 0.94 μm to 0.55 μm
stage 7: from 0.55 μm to 0.34 μm
stage 8 (MOC): <0.34 μm

The respirable fraction (Fine Particle Fraction) is the amount of drug, calculated with respect to the dose delivered, characterized by particles having a median aerodynamic diameter lower than 5.0 μm and is calculated using specific validated software (CITDAS Copley).

The aerodynamic parameters of an inhalation formulation subjected to NGI analysis are expressed in terms of:
Delivered Fraction (DF): i.e. the percentage of the dose of active agent delivered from the mouthpiece of the inhaler, with respect to the loaded dose.
Fine Particle Dose (FPD): theoretically respirable fraction of active ingredient, characterized by an aerodynamic diameter <5.0 μm.
Fine Particle Fraction (FPF): theoretically respirable fraction (aerodynamic diameter <5.0 μm) of active agent expressed as percentage of the amount delivered.
Mass Median Aerodynamic Diameter (MMAD): median aerodynamic diameter of the particles delivered.

Quantitative determination of the active agent in each stage was performed by HPLC using the test method for titer and related substances, the only difference being at solvent level, for which an internal standard (testosterone) was added with the aim of minimizing the analytical error caused by its evaporation during the recovery stage of the NGI test samples. Unlike the analysis method for titer and related substances, in the new solvent testosterone is added at the concentration of ca. 10 μg/ml in the 70/30 methanol/water solution.

The voriconazole content is calculated from the ratio between the area of the active ingredient with respect to the area of the testosterone (retention time 2.6 min) in the sample, with respect to the same ratio in the reference solution at known concentration.

### Characterization of the Powder: Determination of the Solid State by X-Ray Diffractometry and Calculation of the Percentage of Crystallinity.

### X-Ray Diffractometry Measurement

X-ray diffractometry measurements were conducted to determine the solid state of the powder. The crystals diffract the X-rays in a manner characteristic of their structure. For this reason, the X-ray diffractometry technique allows determination of the crystalline or amorphous solid state of the components of the sample.

The instrument used is the Bruker AXS D2-Phaser with LYNXEYE detector, measurement software DIFFRAC.MEASUREMENT CENTER.V7.

The powder samples were arranged in a uniform layer on silicon sample holders with dome with separator, model A100B139 (Steel Airtight Specimen Holder).

The analysis method selected used the following instrument configuration:
Source: copper
Divergence Slit: 0.2 mm
Soller Slit: 4°

The following scanning parameters were used:
Angle range: 4-50° 2 Theta
Step size: 0.03°
Dwell time at each angle: 1 s
Detector aperture: 4 mm
No rotation of the sample

### Calculation of the Crystallinity Percentage

The crystalline nature of the components was measured by comparison with reference structures found in the literature and samples of crystalline raw material.
The Bruker AXS DIFFRAC.TOPAS.V6 software was used to analyze the diffractograms. The diffractograms were loaded into the software and the reference structures in STR format of Voriconazole and Leucine were associated with them, both created from the online CIF files on the Crystallography Open Database website (U.S. Pat. Nos. 2,212,055 and 2,108,011, respectively) with the following changes:
   refinement of the cell parameters
   preferential orientation of 0 0 1 for Leucine and 0 0 2 for Voriconazole.
The following parameters were selected for diffractogram analysis:
   Background: algorithm of order 3 with Chebyshev correction and 1/×Bkg
   Peak shift: sample displacement correction
   Sample Convolutions: absorption correction by fixed sample thickness 0.5 mm
A Peak Phase was added as measure of the amorphous component. The minimum point between the peaks at 19°2 Th and at 21° 2 Th was selected on the graph for each diffractogram.
As this is the reference for the amorphous component, the Crystallite Size L was suggested as 1, leaving the possibility for refinement, while the parameters of position and area of the peak were given fixed settings. This phase was then identified as amorphous for calculation of the degree of crystallinity of the sample.

The fitting was always launched up to the computation limit of the software and accepted within an Rwp value no greater than 15.

The tables below illustrate a series of examples conducted according to the specifications indicated above in order to demonstrate how powders containing voriconazole at high concentrations and with high respirabilities are obtained with a manufacturing method according to the present invention.

In particular, Tables 1 and 2 illustrate the process conditions at which the examples were conducted, while Tables 3 and 4 illustrate the characteristics of the powders obtained with the process according to the invention.

TABLE 1

| | | | | COMP. | | | | |
|---|---|---|---|---|---|---|---|---|
| | | | | MIX | | | | |
| | | COMP. | CONC. | SOLVENT | INLET | OUTLET | | |
| | SPRAY | (%) | SOLUTION | (V/V) | T | T | FEED | YIELD |
| EX. # | DRYER | (VRZ:LEU:TW80) | (% W/W) | ETOH/H₂O | (° C.) | (° C.) | (G/MIN) | (%) |
| 1 | PSD1 | 70/29/1 | 1.0 | 60/40 | 170 | 86 | 20 | 19 |
| (comparison) | | | | | | | | |
| 2 | PSD1 | 70/29/1 | 1.0 | 60/40 | 90 | 44 | 20 | 44 |
| 3 | PSD1 | 70/29/1 | 1.0 | 60/40 | 120 | 60 | 20 | 54 |
| 4 | PSD1 | 55/44/1 | 1.0 | 60/40 | 90 | 44 | 20 | 38 |
| 5 | PSD1 | 85/14/1 | 1.0 | 60/40 | 90 | 44 | 20 | 43 |
| 6 | PSD1 | 85/14/1 | 1.0 | 60/40 | 120 | 60 | 20 | 60 |

TABLE 2

| | | | | COMP. | | | | |
|---|---|---|---|---|---|---|---|---|
| | | | | MIX | | | | |
| | | COMP. | CONC. | SOLVENTE | T | T | | |
| | SPRAY | (%) | SOLUZIONE | (V/V) | INLET | OUTLET | FEED | YIELD |
| ES. # | DRYER. | (VRZ:LEU:TW80) | (% W/W) | ETOH/H₂O | (° C.) | (° C.) | (G/MIN) | (%) |
| 7 | PSD2 | 70/29/1 | 1.0 | 60/40 | 82 | 44 | 120 | 30 |
| 8 | PSD2 | 70/29/1 | 1.0 | 60/40 | 83 | 44 | 120 | 28 |
| 9 | PSD2 | 70/29/1 | 1.0 | 60/40 | 95 | 52 | 120 | 33 |
| 10 | PSD2 | 70/29/1 | 1.0 | 60/40 | 98 | 60 | 100 | 45 |
| 11 | PSD2 | 70/29/1 | 1.0 | 60/40 | 103 | 60 | 120 | 43 |
| 12 | PSD2 | 70/29/1 | 1.0 | 60/40 | 120 | 60 | 160 | 52 |
| 13 | PSD2 | 70/29/1 | 1.5 | 60/40 | 114 | 60 | 140 | 58 |
| 14 | PSD2 | 70/29/1 | 1.0 | 60/40 | 117 | 60 | 160 | 51 |
| 15 | PSD2 | 70/29/1 | 1.0 | 60/40 | 117 | 60 | 160 | 55 |
| 16 | PSD2 | 70/29/1 | 1.0 | 60/40 | 126 | 60 | 180 | 51 |
| 17 | PSD2 | 70/29/1 | 1.0 | 60/40 | 114 | 60 | 140 | 55 |
| 18 | PSD2 | 70/29/1 | 1.0 | 60/40 | 116 | 75 | 100 | 46 |
| 19 | PSD2 | 70/29/1 | 1.0 | 60/40 | 124 | 75 | 120 | 50 |
| 20 | PSD2 | 70/29/1 | 1.0 | 60/40 | 123 | 75 | 120 | 39 |

TABLE 3

| | | | CRYSTAL- | | | |
|---|---|---|---|---|---|---|
| | | | LINITY | VRZ | | |
| EX. | X90 | VMD | XRPD | CONTENT | MMAD | FPF |
| # | (μM) | (μM) | (%) | (%) | (μM) | (%) |
| 1 | 13.0 | 5.5 | 93.9 | 109.0 | 5.1 | 30.5 |
| (comparison) | | | | | | |
| 2 | 5.4 | 2.7 | 93.1 | 102.9 | 3.3 | 73.4 |
| 3 | 7.9 | 3.8 | 93.7 | 106.4 | 3.6 | 67.2 |
| 4 | 4.3 | 2.3 | 94.9 | 104.2 | 3.2 | 75.9 |
| 5 | 4.6 | 2.4 | 92.9 | 104.7 | 3.2 | 72.3 |
| 6 | 6.9 | 3.3 | 93.1 | 104.5 | 4.2 | 56.3 |

TABLE 4

| | | | CRISTAL- | | | |
|---|---|---|---|---|---|---|
| | | | LINITA' | VRZ | | |
| ES. | X90 | VMD | XRPD | CONTENUTO | MMAD | FPF |
| # | (μM) | (μM) | (%) | (%) | (μM) | (%) |
| 7 | 5.4 | 2.9 | 93.7 | 101.3 | 4.3 | 47.4 |
| 8 | 5.4 | 2.9 | 93.7 | 99.5 | 3.8 | 57.2 |
| 9 | 5.6 | 3.0 | 93.7 | 99.8 | 3.8 | 59.3 |
| 10 | 5.0 | 2.7 | 93.9 | 100.1 | 4.0 | 58.9 |
| 11 | 6.0 | 3.1 | 93.8 | 103.0 | 4.3 | 51.5 |
| 12 | 6.6 | 3.3 | | 100.1 | 4.1 | 51.7 |
| 13 | 5.7 | 3.0 | | 99.1 | 4.0 | 53.8 |
| 14 | 5.9 | 3.1 | | 102 | 4.2 | 53.3 |
| 15 | 5.5 | 2.9 | | 99.3 | 4.0 | 58.4 |
| 16 | 6.8 | 3.5 | | 99.5 | 4.2 | 48.5 |
| 17 | 5.6 | 3.0 | | 99.9 | 4.1 | 54.4 |
| 18 | 7.1 | 3.5 | 93.4 | 106.2 | 4.9 | 40.5 |
| 19 | 7.9 | 4.1 | 93.4 | 103.8 | 4.8 | 41.1 |
| 20 | 7.6 | 3.7 | 93.4 | 104.1 | 4.6 | 46.5 |

### Examples 1-2-3

Examples 1, 2, and 3 report formulations containing Voriconazole as active ingredient, having the same percentage composition and obtained by spray drying, drying a hydroalcoholic solution of the components, as described above, at different drying temperatures, using a NIRO PSD1 spray dryer.

The examples highlight the importance of the process temperature, intended as outlet temperature (temperature of the product exiting from the drying chamber), resulting from the combined effects of the drying Temperature (T inlet) and of the feed speed of the solution to be dried (Feed Rate), in order to obtain a formulation of spray-dried Voriconazole with optimal characteristics from the point of view of particle size obtained, of their aerodynamic characteristics and of the homogeneity of the powder from a chemical point of view, determined through the titer of the active ingredient.

Example 1 highlights how a process carried out at high temperatures resulted in a powder characterized by large particles with a diameter corresponding to 90% of the size distribution of 13 μm, only around 30% of which is respirable (FPF 30.5%). In fact, at high temperatures, drying of the single components takes place in different times, resulting in a non-homogeneous powder, in which only particles of active ingredient, which tend to accumulate in the collection cyclone, or only particles of the excipient (Leucine), which instead tend to accumulate in the collection filter, are present, so that the powder accumulated by the cyclone is rich in active ingredient (titer 109%).

A reduction of the inlet temperature to 90° C., corresponding to an outlet temperature of 44° C., allows a reduction in the drying speed of the component with the most tendency to precipitate, so that drying of the components takes place simultaneously, allowing the formation of fine particles (X₉₀=5.4 μm) with a high respirability (FPF 73.4%) in which the active ingredient is distributed uniformly (titer 102.9%). The improvement of the physical, aerodynamic and chemical properties is inversely proportional to the process temperature. (Examples 1-3-2). The yield of the powder is calculated by evaluating the powder collected in the cyclone.

### Example 4

Example 4 reports a formulation of spray dried voriconazole in which the active ingredient is present in a smaller amount with respect to examples 2-3.

Also in this case, a low process temperature results in a formulation characterized by fine particles (X₉₀=4.3 μm) with a high respirability (FPF >75%) and a titer in active ingredient of 104.2%.

### Examples 5-6

Examples 5-6 report formulations of spray dried voriconazole in which the active ingredient is present in a larger amount with respect to examples 2-3.

Also in this case, and hence once again varying the composition of the formulation, the effect of the temperature on the characteristics of the product obtained are in any case evident. In fact, at high temperatures, also in this case, a product characterized by a larger particle size is obtained with respect to the corresponding formulation obtained at low temperatures (X₉₀ 6.9 μm Vs 4.6 μm). Likewise, also the aerodynamic characteristics of the formulation obtained at low temperature are higher (FPF 72.3% vs 56.3%)

### Examples 7-20

Examples 7-20 were obtained starting from a composition similar to examples 2-3 (70% Voriconazole) but operating with a PSD2-Industrial scale spray dryer. Also for this type of spray dryer conditions that apply low process temperatures were set. Inlet temperature 82-130° C. for a feed rate of 100-180 g/min such as to obtain an outlet temperature of the product of 44-75° C.

In particular Examples 7 and 8 has respectively an Inlet temperature of 82 and 83° C., a feed rate of 120 g/min such as to obtain an outlet temperature of the product of 44° C.

Example 9 have an Inlet temperature of 82 and 83° C., a feed rate of 120 g/min such as to obtain an outlet temperature of the product of 52° C.

Examples 10-17 has an Inlet temperature from 98 to 126° C., a feed rate from 100 to 180 g/min such as to obtain an outlet temperature of the product of 60° C.

Examples 18-20 has: an Inlet temperature from 116 to 124° C., a feed rate from 100 to 120 g/min such as to obtain an outlet temperature of the product of 75° C.

With these process conditions it is possible to obtain a spray dried voriconazole powder with a X₉₀ value ranging from 5 to 7.9 μm, and a respirability ranging from 40.5% to 58.9%, the latter for the powder obtained with a lower feed rate (100 g/min) (Ex. 10).

In particular, the powders of Examples 10-17, i.e. the powders obtained with an outlet temperature of 60° C., show a size distribution (X₉₀), an aerodynamic size distribution of the particles (MMAD), and a respirability within the preferred values, associated with a high yield. These examples show how, regardless of the size and of the scale of the equipment used, it is fundamental to maintain low process temperatures in order to obtain a fine spray dried voriconazole powder, respirable and homogeneous in terms of active ingredient content.

These examples also show how the method according to the invention has allowed efficient industrial scaling of the process that did not compromise the physical characteristics and the aerodynamic performance of the voriconazole powders of the present invention.

### Example 21

Example 21 was conducted in order to evaluate the chemical and physical stability of the powders obtained using the method described in the present invention. In particular, the stability at 3 months, 6 months, 12 months and 24 months was evaluated.

A series of powders obtained as described in example 2 set forth above were divided up and packaged in sealed aluminum bags and stored in conditions of 25° C. and 60% relative humidity (RH).

At each interval of time samples were taken and allowed to equilibrate at room temperature, opened and analyzed to evaluate the voriconazole content, the total impurities and some parameters relating to the respirability of the powder, such as X50 (μm), X90 (μm), FPF (%) and MMAD (μm).

Table 5 below provides the stability data according to the description above.

TABLE 5

| | T0 | 3 M | 6 M | 12 M | 24 M |
|---|---|---|---|---|---|
| VRZ content (%) | 101.7 | 100.9 | 99.8 | 99.9 | 100.0 |
| Total impurities (%) | 0.1 | 0.2 | 0.2 | 0.2 | 0.2 |
| X₅₀ (μm) | 1.8 | 1.8 | 1.8 | 1.7 | 1.7 |
| X₉₀ (μm) | 3.8 | 3.9 | 3.8 | 3.7 | 3.7 |
| FPF (%) | 64.9 | 68.1 | 68.0 | 61.0 | 67.8 |
| MMAD (μm) | 3.3 | 3.1 | 3.0 | 3.4 | 2.9 |

1-15. (canceled)

16. A method for manufacturing an inhalable powder comprising leucine and voriconazole or a pharmaceutically active salt thereof, in substantially crystalline form and in an amount greater than 50% by weight with respect to a total amount of the powder, the method comprising:
a) providing a homogeneous solution of voriconazole or its pharmaceutically active salt and leucine in a suitable vehicle;
b) spray drying the powder at an outlet temperature from 40 to 75 degrees Celsius and at a feed rate greater than 10 g/minute; and
c) collecting the powder.

17. The method of claim 16, wherein the powder has a fine particle fraction (FPF) greater than 40%.

18. The method of claim 16, wherein the leucine is present in an amount greater than 10% by weight with respect to the total amount of the powder.

19. The method of claim 16, wherein a surfactant is present in step a).

20. The method of claim 19, wherein the surfactant is present in an amount from 0.2 to 2% by weight with respect to the total amount of the powder.

21. The method of claim 16, wherein at least ninety percent (90%) of a size distribution for the powder is lower than 10 μm.

22. The method of claim 16, wherein the powder has a Mass Median Aerodynamic Diameter (MMAD) equal or lower than 5 μm.

23. The method of claim 16, wherein the voriconazole or its pharmaceutically active salt is present in an amount from 50 to 85% by weight with respect to the total amount of the powder.

24. The method of claim 16, wherein the voriconazole is present in the crystalline solid form in a percentage from 90 to 100% with respect to the total amount of the voriconazole in the powder.

25. The method of claim 16, wherein the surfactant is selected from a group consisting essentially of benzalkonium chloride, cetrimide, sodium docusate, glyceryl monooleate, sorbitan esters, sodium lauryl sulfate, polysorbates, phospholipids, bile salts, polysorbates, polyoxyethylene, and polyoxypropylene block copolymers.

26. The method of claim 16, wherein the vehicle is a hydroalcoholic mixture.

27. The method of claim 16, wherein the leucine is present in crystalline form.

28. The method of claim 16, wherein the feed rate is greater than 15 g/minute.

29. The method of claim 16, wherein the outlet temperature is from 50 to 70° C.

30. An inhalable powder comprising leucine and voriconazole or a pharmaceutically active salt thereof, in substantially crystalline form and in an amount greater than 50% by weight with respect to a total amount of the powder, wherein the inhalable powder is obtained by a process comprising:
a) providing a homogeneous solution of voriconazole or its pharmaceutically active salt and leucine in a suitable vehicle;
b) spray drying the powder at an outlet temperature from 40 to 75 degrees Celsius and at a feed rate greater than 10 g/minute; and
c) collecting the powder.

31. The inhalable powder of claim 30, wherein the powder has a fine particle fraction (FPF) greater than 40%.

32. The inhalable powder of claim 30, wherein the leucine is present in an amount greater than 10% by weight with respect to the total amount of the powder.

33. The inhalable powder of claim 30, wherein a surfactant is present in step a) in an amount from 0.2 to 2% by weight with respect to the total amount of the powder.

34. The inhalable powder of claim 30, wherein at least ninety percent (90%) of a size distribution for the powder is lower than 10 μm.

35. The inhalable powder of claim 30, wherein the powder has a Mass Median Aerodynamic Diameter (MMAD) equal or lower than 5 μm.
